(19) United States
(12) Patent Application Publication
(10) Pub. No.: US 2026/0061056 A1
(43) Pub. Date: Mar. 5, 2026

(54) RINSE SOLUTION FOR A PHOTODYNAMIC DISINFECTION

(71) Applicant: MANA HEALTH TECHNOLOGIES GMBH, Graz (AT)

(72) Inventors: Johannes SMOLLE, Graz (AT); Elisa BÖHM, Graz (AT)

(73) Assignee: MANA HEALTH TECHNOLOGIES GMBH, Graz (AT)

(21) Appl. No.: 18/865,801

(22) PCT Filed: Apr. 17, 2023
(86) PCT No.: PCT/EP2023/059899

§ 371 Date: Nov. 14, 2024

(30) Foreign Application Priority Data
May 16, 2022 (EP) 22173482.5

(51) Int. Cl. A61K 41/00; A61K 9/08; A61K 31/047; A61K 31/17; A61K 31/198; A61K 47/10; A61K 47/36; A61K 47/46

(52) U.S. Cl. CPC A61K 41/0057; A61K 9/08; A61K 31/047; A61K 31/17; A61K 31/198; A61K 47/10; A61K 47/36; A61K 47/46

(57) ABSTRACT

A rinsing solution for photodynamic disinfection, in particular oral disinfection, including, at least one photosensitiser from the group comprising riboflavin 5′-phosphate sodium in a proportion of 0.2 to 0.6 wt. % and a photosensitiser other than riboflavin 5′-phosphate sodium in a proportion of 0.001 to 0.05 wt. %, at least one viscosity modulator from the group comprising glycerol in a proportion of 1.0 to 5.0 wt. % and hyaluronic acid sodium salt in a proportion of 0.01 to 0.3 wt. %, at least one biofilm inhibitor from the group comprising xylitol in a proportion of 7.0 to 25.0 wt. %, L-arginine in a proportion of 0.01 to 0.5 wt. % and urea in a proportion of 0.5 to 3.0 wt. %, with the remainder on a water base.

### CROSS-REFERENCE TO RELATED APPLICATIONS

This application is a National Phase application of International Application No. PCT/EP2023/059899 filed Apr. 17, 2023, which claims priority to the European Patent Application No. 22 173 482.5 filed May 16, 2022, the disclosures of which are incorporated herein by reference.

### TECHNICAL FIELD

The present disclosed subject matter relates to a rinsing solution for photodynamic disinfection, in particular oral disinfection.

### BACKGROUND

In photodynamic disinfection, the interaction of irradiation with light and a photosensitiser destroys harmful microorganisms, e.g. in the mouth or on a mucous membrane, in a wound or a natural or, for example, surgically created body opening, on the surface of the skin or an artificial surface. Mouth disinfection, for example, removes a biofilm on the teeth and gums that is usually formed by bacteria, thereby combatting tooth decay, gingivitis and periodontitis, for example.

When rinsing with the rinsing solution, the photosensitiser penetrates the microorganisms or attaches itself to them and is activated by the irradiation. This generates free radicals, e.g. reactive oxygen species (“ROS”).

The free radicals are neither carcinogenic nor genotoxic to humans and act as a biocide, i.e. as a substance that destroys pathogens in particular, e.g. fungi (including their spores), bacteria, parasites and viruses. Depending on the type of photosensitiser, it either acts as a catalyst in photodynamic disinfection, which generates free radicals from the environment when exposed to light, e.g. from a solution including the photosensitiser; or the photosensitiser itself releases free radicals when exposed to light. The wavelength of the radiation emitted by the radiation source must be matched to the photosensitiser used and its absorption spectrum; it generally ranges from the ultraviolet to the infrared range.

In recent years, many different photosensitisers have been investigated and proposed for the photodynamic disinfection of hands, mouth, teeth, wounds, etc.; for example, US 2016/0051833 A1 mentions chlorophyllin sodium copper salt, riboflavin, allura red, indigo carmine, erioglaucine, tartrazine, chromotrope, true green (“fast green”), lissamine green B, acridine, phenazine, cyanine, phenothiazine, porphyrin, phthalocynine and other photodynamically active substances that may be added to a toothpaste or mouthwash to disinfect the mouth. It also suggests the addition of other ingredients, e.g. caries-inhibiting, antibacterial, abrasive, anti-inflammatory, moisture-regulating substances, flavourings, colourings, etc. and so on. The biofilm reduction achieved with the proposed rinsing solutions or toothpastes with these photosensitisers after irradiation for up to 15 minutes is between 18% and 72% and thus, depending on the photosensitiser, even significantly lower or only slightly higher than with a conventional mouthwash without photosensitiser and without extensive irradiation.

### BRIEF SUMMARY

The aim of the disclosed subject matter is to create a rinsing solution for photodynamic disinfection, in particular oral disinfection, which is significantly more effective in reducing pathogens, in particular biofilm, without being more difficult or complex to use.

This aim is achieved with a rinsing solution for photodynamic disinfection, in particular oral disinfection, which includes at least one photosensitiser from a group of photosensitisers comprising riboflavin 5′-phosphate sodium in a proportion of 0.2 to 0.6 wt. % and/or a photosensitiser other than riboflavin 5′-phosphate sodium in a proportion of 0.001 to 0.05 wt. %, at least one viscosity modulator from a group of viscosity modulators comprising glycerol in a proportion of 1.0 to 5.0 wt. % and/or hyaluronic acid sodium salt in a proportion of 0.01 to 0.3 wt. %, at least one biofilm inhibitor from a group of biofilm inhibitors comprising xylitol in a proportion of 7.0 to 25.0 wt. %, L-arginine in a proportion of 0.01 to 0.5 wt. % and/or urea in a proportion of 0.5 to 3.0 wt. %, with the remainder on a water base. The stated proportions refer to the total rinsing solution.

The present disclosed subject matter is based on the insight that it is not only the choice of photosensitiser or its concentration that has a significant influence on the efficacy of the rinsing solution during irradiation, but that it is precisely the balanced interaction of the various ingredients in the appropriate concentration that produces a surprising, unpredictable increase in efficacy, namely a reduction in bacteria by several orders of magnitude. Thus, the at least one biofilm inhibitor in the specified concentration weakens the integrity of the biofilm by inhibiting the metabolism of the biofilm-forming bacteria, loosening their linkage and/or reducing the surface tension of the biofilm. Due to the at least one viscosity modulator in the specified substance selection and concentration, during rinsing a thin, slightly adherent film with the biofilm inhibitor(s) and photosensitiser(s) is formed on the bacterial structures that are to be reduced, for subsequent irradiation for photodynamic disinfection, whereby the effect of the biofilm inhibitor(s) and photosensitiser(s) is significantly enhanced without the entire rinsing solution having to be kept in the mouth or on the skin, for example, during irradiation; i.e. the rinsing solution is easily and conveniently used to rinse the mouth or other body part before irradiation, for example. The at least one photosensitiser ultimately produces the particularly strong biocidal effect as a result of the formulation of the rinsing solution, i.e. without particularly prolonged or intensive irradiation, i.e. conveniently, simply and without additional effort.

In addition to or instead of the above-mentioned riboflavin 5′-phosphate sodium, various substances with a photodynamic effect may be used as photosensitiser(s), in particular water-soluble photosensitisers, e.g. phycocyanin, but also fat-soluble photosensitisers, e.g. if they are liposomally encapsulated or made soluble in the water base with the aid of emulsifiers, nanoparticle carriers or the like. A single photosensitiser or two or more different photosensitisers may be used.

As is known to a person skilled in the art, the water base may be, for example, water, distilled water or flavoured water or a mixture of water and hydrolate (in particular Eucalyptus or peppermint hydrolate); alternatively, the water base is an aqueous system to which buffers known in the prior art are added for pH stabilisation. For example, to improve solubility, as protection against segregation, to preserve the rinsing solution, etc., other ingredients known in the prior art may be added to the water base, each of which acts, for example, as a buffer system, preservative, carrier system, solubiliser, surfactant or emulsifier and/or promotes micelle formation in the rinsing solution.

It is advantageous if said at least one photosensitiser or one of the (several) photosensitisers is riboflavin 5′-phosphate sodium in a proportion of 0.25 to 0.45 wt. %, e.g. in a proportion of 0.3 wt. %, of the rinsing solution. This photosensitiser has proven to be particularly effective in the rinsing solution, especially in the concentration ranges mentioned. Riboflavin 5′-phosphate sodium is—in addition to its pronounced photodynamic effect—skin caring and as an essential vitamin promotes skin and mucous membrane health and e.g. tooth enamel strength.

Alternatively or additionally, it is favourable if the or one of the photosensitiser(s) is phycocyanin in said proportion of 0.001 to 0.05 wt. %, e.g. in a proportion of 0.005 to 0.025 wt. %, of the rinsing solution. Furthermore, it is favourable if the or one of the photosensitiser(s) is methylene blue in the stated proportion of 0.001 to 0.05 wt. %, e.g. in a proportion of 0.005 to 0.025 wt. %, of the rinsing solution. These two photosensitisers, phycocyanin and methylene blue, have proven to be particularly suitable for use and effective, either on their own or with another photosensitiser, in particular with riboflavin 5′-phosphate sodium.

A particularly suitable rinsing solution is obtained if the or one of the viscosity modulator(s) is glycerol in a proportion of 2.0 to 3.0 wt. %, e.g. in a proportion of 2.5 wt. %, of the rinsing solution. Glycerol not only regulates the viscosity so that the biofilm is effectively and persistently moistened in the specified concentration ranges, but also has a smoothing and moisturising effect on the mucous membrane. This moistening is also favourably influenced if the or one of the viscosity modulator(s) is hyaluronic acid sodium salt in a proportion of 0.02 to 0.1 wt. %, e.g. in a proportion of 0.025 wt. %, of the rinsing solution. In addition to its viscosity-regulating effect, hyaluronic acid sodium salt is also a moisturising agent and has an anti-inflammatory effect in the stated concentration ranges.

Optionally, the or one of the biofilm inhibitor(s) is xylitol in a proportion of 15.0 to 25.0 wt. %, e.g. in a proportion of 15.0 wt. %, of the rinsing solution. Xylitol is a particularly suitable biofilm inhibitor due to its bacteriostatic effect in the specified concentration. In a favourable variant of the rinsing solution, the or one of the biofilm inhibitor(s) is L-arginine in a proportion of 0.01 to 0.045 wt. %, e.g. in a proportion of 0.03 wt. %, of the rinsing solution. L-arginine not only has an antibacterial effect but also an antistatic effect. It is further advantageous if the or one of the biofilm inhibitor(s) is urea in a proportion of 1.0 to 3.0 wt. %, e.g. in a proportion of 1.0 wt. %, of the rinsing solution. Urea attacks the bacterial cell wall and also has an antistatic effect.

Furthermore, it is particularly favourable if the rinsing solution includes at least one penetration enhancer from a group of penetration enhancers comprising ethanol in a proportion of 7.0 to 20.0 wt. %, a mixture of 1,3-propanediol and propanediol in a total proportion of 0.5 to 5.0 wt. % and/or a mixture of Leuconostoc and Radish Root Ferment Filtrate in a total proportion of 0.05 to 0.15 wt. %. The stated proportions refer here to the total rinsing solution and replace a corresponding proportion of the water base. The at least one penetration enhancer improves the penetration of the rinsing solution into the bacteria or other microorganisms in the specified effective concentration ranges and has a general antimicrobial effect.

In an optional embodiment, the or one of the penetration enhancer(s) is ethanol in a proportion of 12.0 to 17.0 wt. %, e.g. in a proportion of 15.0 wt. %, of the rinsing solution. As a solvent, ethanol is not only a penetration enhancer but also has a defoaming, astringent, degreasing, cleansing, antibacterial and viscosity-reducing effect, so that ethanol also interacts with the viscosity modulator(s). It is favourable if the or one of the penetration enhancer(s) is the substance mixture of 1,3-propanediol and propanediol in a total proportion of 1.0 to 5.0 wt. %, e.g. in a total proportion of 3.0 wt. %, of the rinsing solution. 1,3-propanediol and propanediol act as penetration enhancers on the bacterial cell membrane, both individually and especially in a mixture. In an advantageous variant of the rinsing solution, the or one of the penetration enhancer(s) is the substance mixture of Leuconostoc and Radish Root Ferment Filtrate in a total proportion of 0.05 to 0.09 wt. %, e.g. in a total proportion of 0.08 wt. %, of the rinsing solution. This mixture is particularly effective as an antibacterial and antiseborrhoeic agent in the stated concentration ranges.

In order to increase the acceptability of the rinsing solution, it is also favourable if the rinsing solution includes at least one further ingredient from a group of ingredients comprising peppermint extract, ginger extract, xylityl glucoside, 1,4-anhydro-D-xylitol and glucose, the proportion of each of these further ingredients being less than 0.5 wt. % of the rinsing solution. These ingredients in essence promote the flavour and skin compatibility of the rinsing solution and support the effect of all other ingredients in the rinsing solution. Each of the further ingredients replaces a corresponding proportion of the water base in the rinsing solution.

### BRIEF DESCRIPTION OF THE DRAWINGS

The disclosed subject matter is explained in greater detail below with reference to examples and the accompanying drawings. In the drawings:

FIG. 1 shows the absorption spectrum of a photosensitiser included in a rinsing solution according to the disclosed subject matter in a normalised graph over the wavelength;

FIG. 2 shows the efficacy of the photosensitiser of FIG. 1 included in a rinsing solution at different concentrations in a bar chart; and

FIG. 3 shows the efficacy of a rinsing solution according to the disclosed subject matter with the photosensitiser of FIG. 1 in comparison to bacterial growth with and without rinsing solution or with and without irradiation in a bar chart.

### DETAILED DESCRIPTION

According to the present disclosed subject matter, a water-based rinsing solution for photodynamic disinfection, in particular oral disinfection, includes at least one photosensitiser, at least one viscosity modulator for adjusting the viscosity of the rinsing solution, at least one biofilm inhibitor and optionally at least one penetration enhancer. The photosensitiser(s) achieve the photodynamic disinfection effect by destroying pathogenic bacteria, viruses, fungi, parasites, etc. located there when irradiated with a radiation source after rinsing, e.g. the mouth, a mucous membrane, a wound, another natural or, for example, surgically created body opening, a skin surface or an artificial surface with the rinsing solution. For example, inside the mouth, bacteria attach themselves to the teeth and gums as a so-called biofilm. By adjusting the viscosity of the rinsing solution, the viscosity modulator(s) allows/allow a thin film of the rinsing solution to adhere even after rinsing for subsequent irradiation (e.g. in the mouth, particularly on the teeth, gums and biofilm). The biofilm inhibitor(s) support the penetration of the rinsing solution into the biofilm or—generally—into microorganisms and pathogens, which is promoted by the optional penetration enhancer(s).

The choice and concentrations of the individual ingredients and their ratio to each other significantly determine the efficacy of the rinsing solution in photodynamic oral disinfection. For exceptionally high efficacy, the photosensitiser(s) is/are from the group of photosensitisers comprising riboflavin 5′-phosphate sodium in a proportion of 0.2 to 0.6 wt. % (w/w) and/or one or more photosensitisers other than riboflavin 5′-phosphate sodium in a proportion of 0.001 to 0.05 wt. %, i.e. either riboflavin 5′-phosphate sodium in the proportion stated therefor or at least one other photosensitiser in the proportion stated for the other photosensitiser(s), or both. Furthermore, the viscosity modulator(s) is/are from the group of viscosity modulators comprising glycerol in a proportion of 1.0 to 5.0 wt. % and/or hyaluronic acid sodium salt in a proportion of 0.01 to 0.3 wt. %, the biofilm inhibitor(s) is/are from the group of biofilm inhibitors comprising xylitol in a proportion of 7.0 to 25.0 wt. %, L-arginine in a proportion of 0.01 to 0.5 wt. % and/or urea in a proportion of 0.5 to 3.0 wt. %. The remainder of the rinsing solution is a water base, wherein the above-mentioned proportions refer to the entire rinsing solution.

As is known to a person skilled in the art, the water base may be, for example, water, distilled water or flavoured water or a mixture of water and hydrolate, for example eucalyptus hydrolate or peppermint hydrolate (the latter with the CAS registration number 84082-70-2 according to the Chemical Abstract Service, “CAS”, of the American Chemical Society); alternatively, the water base is an aqueous system to which buffer systems known in the prior art are added for the purpose of pH stabilisation (to a value between 4 and 10, e.g. between 5 and 8), for example acids or alkalis including their salts. To improve solubility, as protection against segregation and/or to preserve the rinsing solution, other ingredients known in the prior art may optionally be added to the aqueous system, each of which acts as a buffer system, preservative, carrier system, solubiliser, surfactant or emulsifier and/or promotes micelle formation in the rinsing solution. In addition, the aqueous system may, if necessary, be liposomally encapsulated (for example by phospholipids or fatty acids) and/or may comprise a surfactant-protected or surfactant-free macro-, micro- or nanoemulsion, the latter two being characterised by the particularly favourable property of forming in an optically clear state. Further ingredients known in the prior art may optionally be added to the surfactant-protected or surfactant-free macro-, micro- or nanoemulsion for the purpose of stabilising the pH value, improving the solubility, protecting against segregation and/or improving the bioavailability of the rinsing solution, each of which acts, for example, as a buffer system, preservative, carrier system, solubiliser, surfactant, co-surfactant and/or emulsifier.

The optional penetration enhancer(s) is/are from the group of penetration enhancers comprising ethanol in a proportion of 7.0 to 20.0 wt. % and/or a mixture of 1,3-propanediol and propanediol in a total proportion of 0.5 to 5.0 wt. % and/or a mixture of Leuconostoc and Radish Root Ferment Filtrate in a total proportion of 0.05 to 0.15 wt. %. The stated proportions of the penetration enhancer(s) are in each case based on the total rinsing solution and replace a corresponding proportion of the water base.

The effect of at least one photosensitiser during disinfection is as follows: When rinsing the mouth or another body part or surface to be disinfected, the photosensitiser attaches itself to the microorganisms and pathogens (in the mouth: e.g. to the biofilm) or penetrates them. The photosensitiser is activated by irradiation. This generates free radicals, e.g. reactive oxygen species (“ROS”), which act as a biocide, i.e. as a substance that destroys fungi (including their spores), bacteria, parasites and viruses, for example. The wavelength of the irradiation is matched to the photosensitiser used and its absorption spectrum; it generally ranges from the ultraviolet to the infrared range and may be narrowband or broadband.

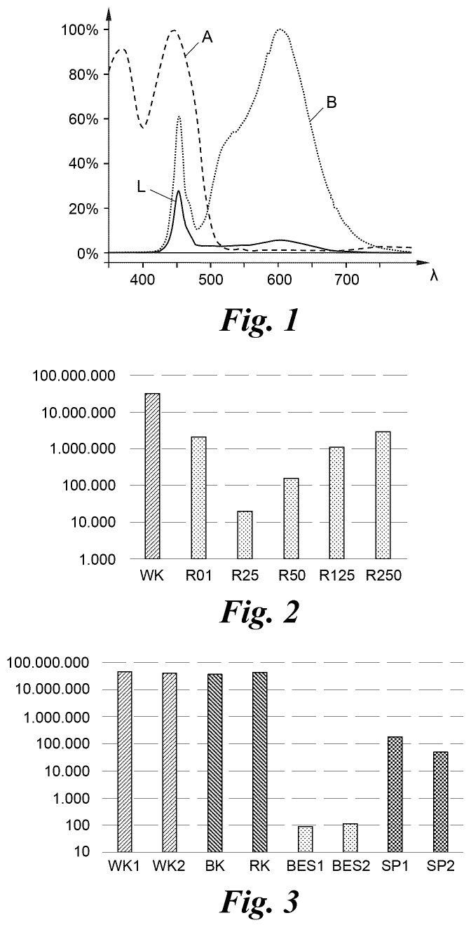

FIG. 1 shows the radiation absorption of a rinsing solution of the aforementioned type with a single photosensitiser (here: riboflavin 5′-phosphate sodium) when irradiated by a radiation source (here: an LED) with a broadband spectrum B (dotted line in FIG. 1), which corresponds to a colour temperature of around 3000 Kelvin. The absorption spectrum A of the photosensitiser used here (dotted line in FIG. 1) has a maximum at a wavelength A of around 445 nm. To activate the photosensitiser, irradiation in the range of this wavelength—or optionally, as in the present example, with a broadband radiation source that also encompasses this range—is therefore effective. The solid line L shows the radiation actually absorbed; it proves that even at wavelengths well beyond the absorption maximum of the photosensitiser (here, for example, at 600 nm), a certain amount of radiation is still absorbed and, albeit less efficiently, free radicals are formed using the energy of the absorbed radiation. If more than one photosensitiser is used, this generally results in several absorption maxima to which the radiation source or its radiation spectrum must be adapted.

In FIG. 2, based on the aforementioned rinsing solution with riboflavin 5′-phosphate sodium as the only photosensitiser, the—in particular photodynamic—effect against a bacterial suspension of the bacterial species Streptococcus mutans (“S. mutans”) is shown as an example: The left comparative bar WK shows the number of colony forming units (CFU) of microorganisms (here: S. mutans) per milliliter (CFU/ml) in the suspension without treatment with rinsing solution and without irradiation in a growth control measurement. The corresponding comparative value was approx. 3.2·10⁷ CFU/ml. The other bars of the graph each show the reduction in the microorganism content when using the (here: single) photosensitiser at different concentrations, including such concentrations that differ from the disclosed subject matter, in the rinsing solution after effective irradiation for 300 s with between 10 and 45 J/cm², in the present case between 17 and 21 J/cm². At a proportion of 0.01 wt. % of the photosensitiser in the rinsing solution, the microorganism content after irradiation was reduced to approx. 2.1·10⁶ CFU/ml, i.e. by more than an order of magnitude (bar R01). The next bar R25 shows a reduction in the microorganism content to approx. 2.0·10⁴ CFU/ml at a photosensitiser concentration of 0.25 wt. % in the rinsing solution, i.e. a reduction by more than 3 orders of magnitude or to less than 1/1000 of the comparative value.

As the other bars R50, R125 and R250 show, the microorganism content increases again with increasing photosensitiser concentration of 0.5 wt. % (R50), 1.25 wt. % (R125) and 2.5 wt. % (R250), i.e. to approx. 1.6·10⁵ CFU/ml, approx. 1.1·10⁶ CFU/ml and approx. 2.9·10⁶ CFU/ml, respectively. This means that despite the higher concentration of the photosensitiser and the higher number of free radicals to be expected as a result, the efficacy of the disinfection is reduced.

In an optional formulation of the rinsing solution, riboflavin 5′-phosphate sodium is the sole photosensitiser in a proportion of 0.25 to 0.45 wt. %, in particular in a proportion of 0.3 wt. %, of the rinsing solution. Other formulations of the rinsing solution include, as an alternative or in addition to riboflavin 5′-phosphate sodium, one or more other photosensitiser(s), in particular phycocyanin and/or methylene blue. The proportion of each photosensitiser different from (“other” than) riboflavin 5′-phosphate sodium is 0.001 to 0.05 wt. %, optionally 0.005 to 0.025 wt. %, of the rinsing solution. Table 1 below lists examples of suitable photosensitisers, each with their identifying CAS registration number:

TABLE 1

| Photosensitiser | CAS number |
|---|---|
| Riboflavin 5′-phosphate sodium | 130-40-5 |
| Phycocyanin | 11016-15-2 |
| Methylene blue | 7220-79-3 |
| Toluidine blue 0 | 92-31-9 |
| Azulene | 275-51-4 |
| Thiamine | 67-03-8 |
| Hypericin | 548-04-9 |
| Curcumin | 458-37-7 |
| Carmine | 1390-65-4 |
| Chlorophyll | 479-61-8 |
| Betanin | 7659-95-2 |
| Rose Bengal | 632-69-9 |
| Eosin Y | 17372-87-1 |
| Erythrosine B | 15905-32-5 |
| Indocyanine green | 3599-32-4 |
| Squaraine | 43134-09-4 |
| Perinaphthenone | 548-39-0 |
| Ruthenium | 7440-18-8 |
| Rhodium | 7440-16-6 |

The viscosity modulators, at least one of which is included in the rinsing solution, and their corresponding CAS numbers are listed in Table 2:

TABLE 2

| Viscosity modulator | CAS number |
|---|---|
| Glycerol | 56-81-5 |
| Hyaluronic acid sodium salt | 9067-32-7 |

In an optional variant, narrower concentration ranges than those listed above are specified for the viscosity modulators: For glycerol a proportion of 2.0 to 3.0 wt. %, e.g. a proportion of 2.5 wt. %, and/or for hyaluronic acid sodium salt a proportion of 0.02 to 0.1 wt. %, e.g. a proportion of 0.025 wt. % of the rinsing solution.

The biofilm inhibitors and their respective CAS numbers are found in Table 3 below:

TABLE 3

| Biofilm inhibitor | CAS number |
|---|---|
| Xylitol | 87-99-0 |
| L-arginine | 74-79-3 |
| Urea | 57-13-6 |

In an optional variant, each of the biofilm inhibitors may also have a narrower concentration range within the concentration ranges specified above. For example, xylitol is optionally included in a proportion of 15.0 to 25.0 wt. %, e.g. in a proportion of 15.0 wt. %, L-arginine is optionally included in a proportion of 0.01 to 0.045 wt. %, e.g. in a proportion of 0.03 wt. %, and urea is optionally included in a proportion of 1.0 to 3.0 wt. %, e.g. in a proportion of 1.0 wt. %, in the rinsing solution.

Table 4 lists the optional penetration enhancers together with the corresponding CAS numbers:

TABLE 4

| Penetration enhancer | CAS number |
|---|---|
| Ethanol | 64-17-5 |
| 1,3-Propanediol/Propanediol | 504-63-2/ |
| | 26264-14-2 |
| Leuconostoc/Radish Root | 1686112-10-6/ |
| Ferment Filtrate | 84775-94-0 |

In an optional embodiment of the rinsing solution, the penetration enhancers each have a narrower concentration range compared to their concentration ranges indicated above. Ethanol may be included, for example, in a proportion of 12.0 to 17.0 wt. %, e.g. in a proportion of 15.0 wt. %, the mixture of 1,3-propanediol and propanediol in a total proportion of 1.0 to 5.0 wt. %, e.g. in a total proportion of 3.0 wt. %, and the mixture of Leuconostoc and Radish Root Ferment Filtrate in a total proportion of 0.05 to 0.09 wt. %, e.g. in a total proportion of 0.08 wt. %, in the rinsing solution.

The two ingredients of the two mixtures mentioned, 1,3-propanediol and propanediol on the one hand and Leuconostoc and Radish Root Ferment Filtrate on the other hand, have any desired ratios to each other within the respective mixtures; both mixtures are available ready-made, but they may also be added to the rinsing solution in ratios other than those available ready-made, in particular up to 100% of one or other of the two ingredients of the respective mixtures.

Optionally, the rinsing solution may also include further ingredients that are primarily used for flavour and skin compatibility. The following Table 5 shows some examples of these ingredients again with the respective CAS number (if available) and their respective proportions by weight (in wt. %):

TABLE 5

| | | Proportion by |
|---|---|---|
| Further ingredient | CAS number | weight |
| Peppermint extract | 84082-70-2 | <0.4 |
| Ginger extract | 84696-15-1 | <0.4 |
| Xylityl glucoside | — | <0.5 |
| 1,4-anhydro-D-xylitol | 53448-53-6 | <0.34 |
| Glucose | 50-99-7 | <0.05 |

The water base may optionally also be mixed with additional ingredients that may be assigned to the defined groups of viscosity modulators, biofilm inhibitors, penetration enhancers and support their respective efficacy, or such ingredients that are already used in conventional mouthwashes and/or toothpastes known in the prior art and are used for the purpose of enamel protection or for the care and pain relief of the mucous membrane.

It is understood that the proportion of the water base of the rinsing solution is reduced by the respective proportion by weight of each further (or additional) ingredient included.

The following are some examples of the rinsing solution of the present disclosure.

### Example 1

The rinsing solution according to Example 1 was composed as shown in the following Table 6 (proportions by weight in wt. %):

TABLE 6

| | Proportion by weight |
|---|---|
| Photosensitiser: | |
| Riboflavin 5′-phosphate sodium | 0.3 |
| Viscosity modulators: | |
| Glycerol | 2.5 |
| Hyaluronic acid sodium salt | 0.025 |
| Biofilm inhibitors: | |
| Xylitol | 15.0 |
| L-arginine | 0.03 |
| Urea | 1.0 |
| Penetration enhancers: | |
| Ethanol | 15.0 |
| 1,3-Propanediol/Propanediol | 3.0 |
| Leuconostoc/Radish Root | 0.08 |
| Ferment Filtrate | |
| +remainder water base | 63.065 |

FIG. 3 shows the effect of the rinsing solution according to Example 1 using two series of tests. The two bars WK1 and WK2 again show growth control measurements of colony-forming units of the microorganisms in a bacterial suspension (here: again S. mutans): In the first test series, a value of 4.5·10⁷ CFU/ml (WK1) was measured; in the second test series a (similar) value of 4.0·10⁷ CFU/ml (WK2) was measured.

For the first test series, an irradiation control measurement was also carried out after a two-minute incubation in the dark and irradiation according to FIG. 2 with a radiation source according to FIG. 1 without rinsing solution, which led to a slight reduction of the measured value to 3.7·10⁷ CFU/ml according to the bar BK in FIG. 3. Another measurement (bar RK) involved a 1:1 mixing of the bacterial suspension with the rinsing solution according to Example 1, which resulted in a value of 4.3·10⁷ CFU/ml after seven minutes of incubation and without irradiation.

Furthermore, two comparative measurements with rinsing solution and irradiation were carried out for the first test series, with an incubation time of 120 seconds and an irradiation time of 300 seconds. The results of these measurements are shown in bars BES1 and BES2; the corresponding measured values were 9.0·10¹ CFU/ml and 1.1·10² CFU/ml, i.e. around 5.5 orders of magnitude less than the value (WK1 or WK2) of the growth control or around 1/500000.

For the comparative second test series, two different conventional mouthwashes (without photodynamic properties) were incubated in the dark for the prescribed 30 seconds; the following measurements resulted in values of 1.8·10⁵ CFU/ml (bar SP1) for the first mouthwash and 5.0·10⁴ CFU/ml (bar SP2) for the second mouthwash.

Accordingly, the rinsing solution with photodynamic effect according to Example 1 is around 1000 times more effective than conventional mouthwashes.

### Example 2

According to Example 2 or Table 7, an alternative formulation included (proportions by weight in wt. %)

TABLE 7

| | Proportion by weight |
|---|---|
| Photosensitiser: | |
| Riboflavin 5′-phosphate sodium | 0.25 |
| Viscosity modulators: | |
| Glycerol | 1.5 |
| Hyaluronic acid sodium salt | 0.025 |
| Biofilm inhibitors: | |
| Xylitol | 8.5 |
| L-arginine | 0.015 |
| Urea | 0.5 |
| Penetration enhancers: | |
| Ethanol | 15.0 |
| 1,3-Propanediol/Propanediol | 1.5 |
| +remainder water base | 72.71 |

### Example 3

A good effect was also achieved with a rinsing solution according to Example 3 or Table 8, which included additional ingredients mainly to improve the flavour (proportions by weight in wt. %):

TABLE 8

| | Proportion by weight |
|---|---|
| Photosensitiser: | |
| Riboflavin 5′-phosphate sodium | 0.3 |
| Viscosity modulators: | |
| Glycerol | 2.5 |
| Hyaluronic acid sodium salt | 0.025 |
| Biofilm inhibitors: | |
| Xylitol | 15.0 |
| L-arginine | 0.03 |
| Urea | 1.0 |
| Penetration enhancers: | |
| Ethanol | 15.0 |
| 1,3-Propanediol/Propanediol | 3.0 |
| Leuconostoc/Radish Root | 0.08 |
| Ferment Filtrate | |
| other ingredients: | |
| Peppermint extract | 0.2 |
| Ginger extract | 0.2 |
| Xylityl glucoside | 0.3 |
| 1,4-anhydro-D-xylitol | 0.15 |
| Glucose | 0.04 |
| +remainder water base | 62.175 |

### Example 4

Example 4 included a simplified formulation according to the following Table 9 (proportions by weight in wt. %):

TABLE 9

| | Proportion by weight |
|---|---|
| Photosensitiser: | |
| Riboflavin 5′-phosphate sodium | 0.25 |
| Viscosity modulators: | |
| Glycerol | 2.7 |
| Biofilm inhibitors: | |
| Xylitol | 18.0 |
| Urea | 2.0 |
| Penetration enhancers: | |
| Ethanol | 12.5 |
| 1,3-Propanediol/Propanediol | 4.5 |
| +remainder water base | 60.05 |

### Example 5

Example 5 shows another formulation in Table 10 (proportions by weight in wt. %):

TABLE 10

| | Proportion by weight |
|---|---|
| Photosensitiser: | |
| Phycocyanin | 0.05 |
| Viscosity modulators: | |
| Glycerol | 3.0 |
| Hyaluronic acid sodium salt | 0.05 |
| Biofilm inhibitors: | |
| Xylitol | 22.4 |
| Urea | 1.7 |
| Penetration enhancer: | |
| Ethanol | 16.5 |
| +remainder water base | 56.3 |

### Example 6

Example 6 corresponded to a formulation listed in the following Table 11 (proportions by weight in wt. %):

TABLE 11

| | Proportion by weight |
|---|---|
| Photosensitiser: | |
| Phycocyanin | 0.01 |
| Methylene blue | 0.012 |
| Viscosity modulators: | |
| Hyaluronic acid sodium salt | 0.08 |
| Biofilm inhibitors: | |
| Xylitol | 12.5 |
| Urea | 3.0 |
| Penetration enhancer: | |
| Ethanol | 13.0 |
| +remainder water base | 71.389 |

It is understood that the disclosed formulations and weight proportions are subject to production-related fluctuations or measurement inaccuracies, the magnitude of which depends in particular on the production and measurement methods used; thus, depending on the method, deviations from the stated proportions by weight in the range of typically up to +/−10% of the stated proportions by weight (not of the total rinsing solution) are possible and included.

The disclosed subject matter is not limited to the exemplary embodiments shown and described, but encompasses all variants, modifications and combinations thereof which fall within the scope of the appended claims.

What is claimed is:

1. A rinsing solution for photodynamic disinfection, including,
at least one photosensitiser from a group of photosensitisers comprising riboflavin 5′-phosphate sodium in a proportion of 0.2 to 0.6 wt. % and/or a photosensitiser other than riboflavin 5-phosphate sodium in a proportion of 0.001 to 0.05 wt. %,
at least one viscosity modulator from a group of viscosity modulators comprising glycerol in a proportion of 1.0 to 5.0 wt. % and/or hyaluronic acid sodium salt in a proportion of 0.01 to 0.3 wt. %,
at least one biofilm inhibitor from a group of biofilm inhibitors comprising xylitol in a proportion of 7.0 to 25.0 wt. %, L-arginine in a proportion of 0.01 to 0.5 wt. % and/or urea in a proportion of 0.5 to 3.0 wt. %,
with a remainder on a water base.

2. The rinsing solution according to claim 1, wherein the photosensitiser or one of the photosensitisers is riboflavin 5′-phosphate sodium in a proportion of 0.25 to 0.45 wt % of the rinsing solution.

3. The rinsing solution according to claim 1, wherein the photosensitiser or one of the photosensitisers is phycocyanin in a proportion of 0.005 to 0.025 wt. % of the rinsing solution.

4. The rinsing solution according to claim 1, wherein the photosensitiser or one of the photosensitisers is methylene blue in a proportion of 0.005 to 0.025 wt. % of the rinsing solution.

5. The rinsing solution according to claim 1, wherein the viscosity modulator or one of the viscosity modulators is glycerol in a proportion of 2.0 to 3.0 wt. % of the rinsing solution.

6. The rinsing solution according to claim 1, wherein the viscosity modulator or one of the viscosity modulators is hyaluronic acid sodium salt in a proportion of 0.02 to 0.1 wt. % of the rinsing solution.

7. The rinsing solution according to claim 1, wherein the biofilm inhibitor or one of the biofilm inhibitors is xylitol in a proportion of 15.0 to 25.0 wt. % of the rinsing solution.

8. The rinsing solution according to claim 1, wherein the biofilm inhibitor or one of the biofilm inhibitors is L-arginine in a proportion of 0.01 to 0.045 wt. % of the rinsing solution.

9. The rinsing solution according to claim 1, wherein the biofilm inhibitor or one of the biofilm inhibitors is urea in a proportion of 1.0 to 3.0 wt. % of the rinsing solution.

10. The rinsing solution according to claim 1, further including at least one penetration enhancer from a group of penetration enhancers comprising ethanol in a proportion of 7.0 to 20.0 wt. %, a mixture of 1,3-propanediol and propanediol in a total proportion of 0.5 to 5.0 wt. % and/or a mixture of Leuconostoc and Radish Root Ferment Filtrate in a total proportion of 0.05 to 0.15 wt. %, wherein the stated proportions are based in each case on the total rinsing solution and replace a corresponding proportion of the water base.

11. The rinsing solution according to claim 10, wherein the penetration enhancer or one of the penetration enhancers is ethanol in a proportion of 12.0 to 17.0 wt. % of the rinsing solution.

12. The rinsing solution according to claim 10, wherein the penetration enhancer or one of the penetration enhancers is the mixture of 1,3-propanediol and propanediol in a total proportion of 1.0 to 5.0 wt. % of the rinsing solution.

13. The rinsing solution according to claim 10, wherein the penetration enhancer or one of the penetration enhancers is the mixture of Leuconostoc and Radish Root Ferment Filtrate in a total proportion of 0.05 to 0.09 wt. % of the rinsing solution.

14. The rinsing solution according to claim 1, further including at least one further ingredient from a group of ingredients comprising peppermint extract, ginger extract, xylityl glucoside, 1,4-anhydro-D-xylitol and/or glucose, wherein a proportion of any of these further ingredients is less than 0.5 wt. % of the rinsing solution and replaces a corresponding proportion of the water base.

15. The rinsing solution according to claim 1, wherein the photosensitiser or one of the photosensitisers is riboflavin 5′-phosphate sodium in a proportion of 0.3 wt. % of the rinsing solution.

16. The rinsing solution according to claim 1, wherein the viscosity modulator or one of the viscosity modulators is glycerol in a proportion of 2.5 wt. % of the rinsing solution.

17. The rinsing solution according to claim 1, wherein the viscosity modulator or one of the viscosity modulators is hyaluronic acid sodium salt in a proportion of 0.025 wt. % of the rinsing solution.

18. The rinsing solution according to claim 1, wherein the biofilm inhibitor or one of the biofilm inhibitors is xylitol in a proportion of 15.0 wt. % of the rinsing solution.

19. The rinsing solution according to claim 1, wherein the biofilm inhibitor or one of the biofilm inhibitors is L-arginine in a proportion of 0.03 wt. % of the rinsing solution.

20. The rinsing solution according to claim 1, wherein the biofilm inhibitor or one of the biofilm inhibitors is urea in a proportion of 1.0 wt. % of the rinsing solution.
